(19) United States
(12) Patent Application Publication
(10) Pub. No.: US 2026/0225883 A1
(43) Pub. Date: Aug. 6, 2026

(54) Hydrogen Energy Storage Via Plasma-Based Technology

(71) Applicants: Shayan S. Niknezhad, Bryan, TX (US); Efstratios Pistikopoulos, College Station, TX (US); David Staack, College Station, TX (US)

(72) Inventors: Shayan S. Niknezhad, Bryan, TX (US); Efstratios Pistikopoulos, College Station, TX (US); David Staack, College Station, TX (US)

(73) Assignee: The Texas A&M University System, College Station, TX (US)

(21) Appl. No.: 19/142,536

(22) PCT Filed: Dec. 22, 2023
(86) PCT No.: PCT/US2023/085734

§ 371 Date: Jun. 23, 2025

Related U.S. Application Data
(60) Provisional application No. 63/434,969, filed on Dec. 23, 2022.

(51) Int. Cl. C01B 3/34; B01D 53/22; B01J 19/08; C01B 3/501; C07C 2/80

(52) U.S. Cl. CPC C01B 3/342; B01D 53/228; B01D 53/229; B01J 19/088; C01B 3/501; C07C 2/80; B01D 2256/16; B01D 2257/7022; B01J 2219/00033; B01J 2219/0805; B01J 2219/0869; B01J 2219/0896; C01B 2203/0211; C01B 2203/041; C01B 2203/048; C01B 2203/1241

(57) ABSTRACT

Provided herein are systems and processes for hydrogen production and storage. The systems have three main components that are a plasma-based reformer, a membrane and separation component and a gas compressor(s) that form a continuous fluid loop. The systems also may have a storage system for hydrogen energy storage. In the process natural gas is reformed in the plasma-based reformer into a mix of hydrogen and low-molecular weight hydrocarbons, flowed into the membrane and separation component where the hydrogen is separated out of the mix and stored and the hydrocarbons are recirculated via the gas compressors to the plasma-based reformer.

### CROSS-REFERENCE TO RELATED APPLICATIONS

This international application claims benefit of priority under 35 U.S.C. § 119(e) of provisional application U.S. Ser. No. 63/434,969, filed Dec. 23, 2022, the entirety of which is hereby incorporated by reference.

### BACKGROUND OF THE INVENTION

### Field of the Invention

The present invention relates to the fields of gas separation and energy storage. More particularly, the present invention relates to plasma-based methods for separating hydrogen from natural gas and storing the separated products.

### Description of the Related Art

Some technologies for producing hydrogen are available today. The most common method is Steam Methane Reforming (SMR), an energy-intensive thermal process that emits carbon dioxide and requires catalysis. Moreover, the SMR process plants must be large enough to make them profitable.

Electrolytic processes use electricity to convert water to hydrogen. These processes do not emit carbon dioxide, but they require water as the feedstock and should be used in scales to make them profitable. Direct solar water splitting and biological methods are two processes in the early research stage. As reported by the Department of Energy, the cost of producing hydrogen through solar water splitting and biological processes is high, $8.25 and $9.58 per kilogram, respectively.

Thus, there is a need in the art for hydrogen production and storage processes that do not consume water, do not emit carbon dioxide, nitrogen oxides (NOx) or sulfur oxides (Sox) and can save the majority of wasted electricity produced by renewable sources. Particularly, there is a need for a modularized process to reform natural gas to hydrogen and co-products using electricity at ambient temperature and pressure. The present invention fulfills this long-standing need and desire in the art.

### SUMMARY OF THE INVENTION

The present invention is directed to a system for producing hydrogen. The system has a gas reformer component, a membrane-based gas separation component in fluid connection with the gas reformer component and means for recirculating non-hydrogen gases in fluid connection with the gas reformer and the membrane-based gas separation component. The present invention is direct further to a related system further comprising a storage component in fluid connection with the membrane-based gas separation component.

The present invention also is directed to a method for producing hydrogen. In the method in step a) methane is fed into the plasma-based reformer component described herein to produce a mixture of hydrogen gas and hydrocarbon gases. In step b) the mixture is passed through the membrane-based gas separation component to separate the hydrogen gas from the hydrocarbon gases. The present invention is direct to a related method further comprising a step c) of flowing the hydrogen gas into a storage container. The present invention is directed further to another related method further comprising a step d) of flowing the hydrocarbon gases to the plasma-based reformer component and repeating steps a) to d) at least once in step e).

The present invention is directed further to a modular system for producing hydrogen energy from natural gas. The modular system is a continuous fluid loop comprising a plasma-based reformer, a membrane and separation component and at least one compressor. The plasma-based reformer is operable to reform natural gas into a mixture of hydrogen and low molecular weight hydrocarbon products, the membrane and separation component is operable to separate the mixture into a permeate consisting of the hydrogen and a retentate comprising the low molecular weight hydrocarbons and the at least one compressor is operable to recirculate the retentate to the plasma-based reformer. The present invention is directed to a related modular system further comprising a storage system in fluid connection therewith, said storage system operable to store the hydrogen as an energy source.

The present invention is directed further still to a continuous flow process for producing hydrogen energy. In the method in step a) natural gas is flowed into a plasma-based reformer. In step b) a plasma is generated within the plasma-based reformer to produce hydrogen gas and low molecular weight hydrocarbons from the natural gas. In step c) the hydrogen gas and low molecular weight hydrocarbons are flowed into a separation component to separate the hydrogen gas from the low molecular weight hydrocarbons where hydrogen gas is flowed through a membrane disposed therein. In step d) the low molecular weight hydrocarbons retained in the separation component are returned to the plasma-based reformer. Steps a) to d) are repeated in step e). The present invention is direct to a related method further comprising storing the hydrogen gas as an energy source.

Other and further aspects, features, benefits, and advantages of the present invention will be apparent from the following description of the presently preferred embodiments of the invention given for the purpose of disclosure.

### BRIEF DESCRIPTION OF THE FIGURES

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIG. 1 is a schematic of the plasma-based hydrogen production system.

FIG. 2 is a schematic of the methane to hydrogen plasma reactor.

FIG. 3 compares the H₂ permeability versus H₂/CH₄ selectivity for previous developed membranes and the designed membrane provided herein.

FIG. 4 is a schematic of the connected external DC electric circuit to electrodes for creating a plasma medium.

FIGS. 5A-5D show the effect of increasing the input pulse voltages at 7, 8 and 10 KHZ of pulse repetition frequency (PRF) (FIG. 5A), for methane conversion (FIG. 5B), for H₂ production (FIG. 5C), H₂ yield (kg/kJ) versus input voltage (FIG. 5D).

FIGS. 6A-6C show the percentage of products after reforming when the input voltage is stabled (=28 kV), and pulse repetition frequency (PRF) is at (a) 10 kHz (FIG. 6A), (b) 8 kHz (FIG. 6B), and kHz (FIG. 6C) demonstrating that changing input voltage has less effect on producing co-products and that maximum H2 production was achieved at the highest voltage and PRF.

FIGS. 7A-7B show the effect of changing the pulse repetition frequency (PRF) products at 28 kV and 1.1 L/min flow (FIG. 7A) and the effect of increasing PRF at various input voltages (FIG. 7B) demonstrating that increasing repetition frequency increases methane conversion and H₂ production, and selectivity.

FIGS. 8A-8B show the specific energy input (SEI) vs input voltage (kV) (FIG. 8A) and SEI (kJ/kg) vs pulse repetition frequence (PRF) (FIG. 8B) demonstrating that SEI increases more by the PRF than the input voltage.

FIGS. 9A-9B show power vs. methane conversion (FIG. 9A) and hydrogen production at 10 kHz, 8 kHz, and 7 kHz of pulse repetition frequency (PRF) (FIG. 9B) demonstrating that maximum hydrogen production (=34.89%) are achieved at a 30 W input power.

FIGS. 10A-10B show methane conversion and H₂ production rate vs. power at 1.1 L/min flow demonstrating that both methane conversion (FIG. 10A) and H₂ production (FIG. 10B) increase by the input power (W).

FIGS. 11A-11B illustrate H₂ yield (kg/kJ) and H₂ selectivity versus power (W) to compare the hydrogen mass production per unit of energy (kJ) at various input voltages and pulse repetition frequencies at a flow rate of 1.1 L/min.

FIGS. 12A-12F show the effect of flow rate (L/min) on the products, including H₂, C₂H₂, C₂H₄+C₂C₆, C₃, and C₄ at 30 kV and 10 kHz (FIG. 12A) 30 KV and 8 kHz (FIG. 12B) 8 kV and 10 KHz (FIG. 12C), 28 kV and 8 kHz (FIG. 12D), 26 kV and 6 KHz (FIG. 12E), 30 KV and 7 kHz (FIG. 12F).

FIG. 13 shows the H₂ yield (kg/kJ) versus power at two flow rates: 2.2 L/min and 1.1 L/min demonstrating that a higher yield is achieved at a 2.2 L/min flow.

FIGS. 14A-14B show the logarithmic density number for H₂, CH₄, C₂H₂, and C₂H₄ (FIG. 14A) and (b) H, CH, CH₂, and CH₃ (FIG. 14B) demonstrating the number of densities for CH, CH₂, and CH₃ radicals increases close to the plasma discharge electrode.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” are understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, “consists of” and its variations, such as “consisting of” are understood to imply the inclusion of a stated item, element or step or group of items, elements or steps but excluding any other item, element or step or group of items, elements or steps.

As used herein, the term “includes” or “including” is used herein to mean “including, but not limited to”. The terms “includes”, “including” and “including but not limited to” are used interchangeably.

As used herein, the terms “plasma-based reformer”, “nanoseconds plasma-based reformer” and “reformer” are used interchangeably.

As used herein, the terms “membrane-based gas separation component”, “membrane and separation component” and “membrane” are used interchangeably.

As used herein, the terms “compressor” in the singular or plural and “gas compressor” in the singular or plural are used interchangeably.

As used herein, the term “permeate” refers to hydrogen gas, produced in the plasma-based reformer, after permeating through the gas membrane separate from the light hydrocarbon products.

As used herein, the term “retentate” refers to at least those light or low molecular weight hydrocarbon gas products, produced in the plasma-based reformer, retained in the gas membrane and subsequently returned to the plasma-based reformer.

In one embodiment of the present invention, there is provided a system for producing hydrogen, comprising a gas reformer component; a membrane-based gas separation component in fluid connection with the gas reformer component; and means for recirculating non-hydrogen gases in fluid connection with the gas reformer and the membrane-based gas separation component. Further to this embodiment, the system comprises a storage component in fluid connection with the membrane-based gas separation component.

In both embodiments, the gas reformer may be a plasma-based reformer configured to discharge of a plurality of nanosecond pulses of electricity to create a non-thermal plasma. In an aspect thereof each nanosecond pulse of electricity may have a pulse width of about 3 ns with an energy input of 3 mJ or less. Also, in both embodiments, the membrane-based gas separation component comprises a plurality of nanotubes therein configured to flow gases therethrough. In addition, the means for recirculating non-hydrogen gases may be at least one compressor.

In another embodiment of the present invention, there is provided a method for producing hydrogen, comprising a) feeding methane into the plasma-based reformer component of claim 1 to produce a mixture of hydrogen gas and hydrocarbon gases; and b) passing the mixture through the membrane-based gas separation component to separate the hydrogen gas from the hydrocarbon gases. Further to this embodiment, the method may comprise after step b) a step c) of flowing the hydrogen gas into a storage container. In another further embodiment, the method may comprise a step d) of flowing the hydrocarbon gases to the plasma-based reformer component; and a step e) of repeating steps a) to d) at least once. In all embodiments, the hydrocarbon gases comprise C₂H₂ hydrocarbons, C₂H₆ hydrocarbons or C₃ hydrocarbons or a combination thereof.

In yet another embodiment of the present invention, there is provided a modular system for producing hydrogen energy from natural gas, comprising, in a continuous fluid loop, a plasma-based reformer operable to reform natural gas into a mixture of hydrogen and low molecular weight hydrocarbon products, a membrane and separation component operable to separate the mixture into a permeate consisting of the hydrogen and a retentate comprising the low molecular weight hydrocarbons; and at least one compressor operable to recirculate the retentate to the plasma-based reformer. Further to this embodiment, the modular system comprises a storage system in fluid connection therewith, said storage system operable to store the hydrogen as an energy source.

In both embodiments, the plasma-based reformer may comprise a plasma reactor in operable communication with an electric power supply. Also, in both embodiments the membrane and separation component may comprise a plurality of nanotubes operable to effect separation of the mixture. In addition, the retentate may comprise C₂H₂ hydrocarbons, C₂H₆ hydrocarbons or C₃ hydrocarbons or a combination thereof.

In yet another embodiment of the present invention, there is provided a continuous flow process for producing hydrogen energy, comprising a) flowing natural gas into a plasma-based reformer; b) generating a plasma within the plasma-based reformer to produce hydrogen gas and low molecular weight hydrocarbons from the natural gas; c) flowing the hydrogen gas and low molecular weight hydrocarbons into a separation component to separate the hydrogen gas from the low molecular weight hydrocarbons, said hydrogen gas flowing through a membrane disposed therein; d) returning the low molecular weight hydrocarbons retained in the separation component to the plasma-based reformer; and e) repeating steps a) to d). Further to this embodiment, the continuous flow process comprises storing the hydrogen gas as an energy source.

In both embodiments, the plasma may be a non-thermal plasma generated via nanosecond pulses of electricity. In an aspect thereof, each of the plurality of nanosecond pulses of electricity has a pulse width of about 3 ns and an energy input of 3 mJ or less. Also in both embodiments, the hydrocarbon gases may comprise C₂H₂ hydrocarbons, C₂H₆ hydrocarbons or C₃ hydrocarbons or a combination thereof.

Provided herein are systems and processes, for example, modularized systems and processes, to convert natural gas to hydrogen. Generally, natural gas, such as methane, passes through a nanoseconds plasma-based reformer and converts to hydrogen. After the feedstock passes by the reformer, products enter the membrane to separate H₂ from light hydrocarbons including CH₄, C₂ₛ, and C₃ₛ. H₂ is separated from the other molecules through the membrane, and the retentate gases from the membrane return to the plasma reformer by gas compressors (pumps). The system comprises three main parts, nanoseconds plasma-based reformer, membrane and separation part, and at least one compressor for recirculating gas through the system. The system may further comprise in a fluid connection means for storing the produced hydrogen and the light hydrocarbon gases, such as but not limited to, storage tanks. The system may be used as a standalone device for producing hydrogen or as a hydrogen energy storage device.

More, particularly, the systems and processes provided herein are effective to:
1) store energy in the form of hydrogen,
2) produce hydrogen as a clean source of energy (combusting hydrogen produces water) via the plasma-based reformer to meet future demand using low energy input,
3) utilize waste electricity from wind turbine farms that cannot currently be stored efficiently to produce hydrogen and to save the majority of the wasted energy (electrical energy) in the form of hydrogen,
4) reach the net zero carbon dioxide emissions target because no oxygen is involved in the process and, thus, there would be no carbon dioxide as either product or direct emission,
5) be used in any location which has natural gas to meet the future hydrogen demand, and
6) produce hydrogen using standard electric plugs.

It is contemplated that the modularized system and process may be useful as:
1) energy storage,
2) a hydrogen reformer,
3) a home energy refueling system, such as refueling hydrogen vehicles,
4) a neighborhood energy station (hydrogen fuel station),
5) on-site hydrogen production for power generation, in places such as, but not limited to, airports for future hydrogen fuel-based airplanes and emergency power generation;
6) a means to reduce flaring natural gas, and
7) a source of hydrogen needed to produce the majority of chemicals.

The following examples are given for the purpose of illustrating various embodiments of the invention and are not meant to limit the present invention in any fashion.

### Example 1

### Hydrogen Production Via Methane Reforming

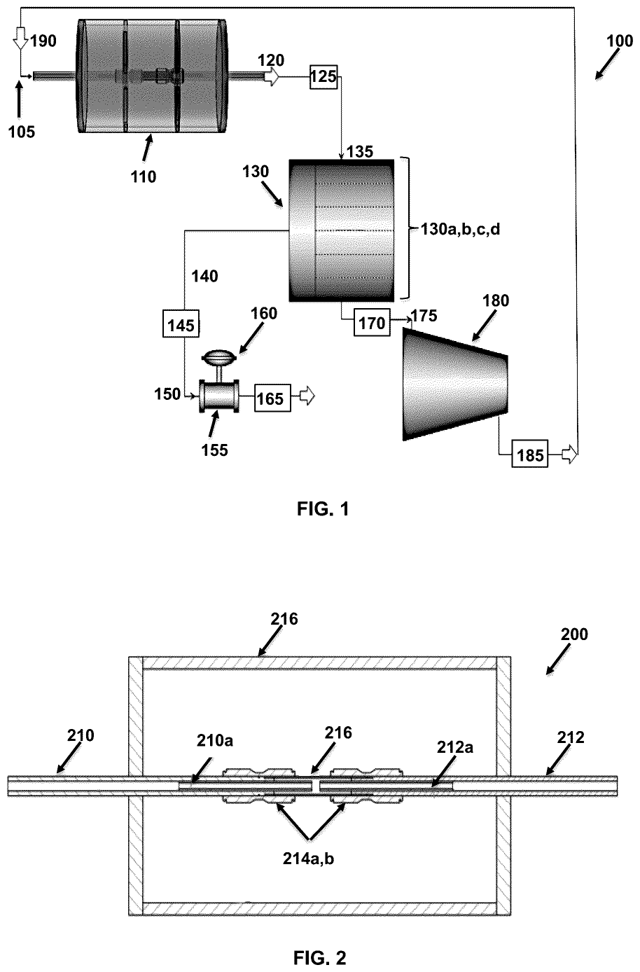

FIG. 1 shows the process diagram 100 for producing hydrogen using non-thermal plasma-based methane reforming. The hydrogen production process is as follows: first, natural gas, i.e., methane 105 as the feedstock, passes through the plasma-based reformer, i.e., nanoseconds plasma-based reformer 110. After the feedstock passes through the reformer at 120, products 125 enter at 135 a membrane 130 with a plurality of nanotubes represented by 130a,b,c,d to separate H₂ from light hydrocarbons. The products include a mix of CH₄, C₂ₛ, and C₃ₛ. H₂ is separated at 140 from the other molecules as a permeate 145 through the membrane and is flowed along 150 to storage 155 or released via valve 160 at 165. The retentate gases 170 comprising the mix of products from the membrane are flowed along 175 to one or more gas compressors represented by 180 and are recirculated at 185 via the gas compressors to the plasma reformer at 190.

Particularly, the three main components of the system are the nanoseconds plasma-based reformer 110, membrane and separation component 130, and at least one compressor or gas compressor 180 for recirculating gas through the system. The nanosecond plasma-based reformer comprises or may consist only of two main components: a plasma reactor and an electric power supply (see FIG. 2).

### Plasma Reactor Design

FIG. 2 shows a schematic of the plasma reactor assembly 200. Two metal tubes 210, 212 are used as electrodes and channels to deliver and eject the gases from the plasma reactor. The anode tube 210a delivers gas and is connected to a high-voltage power supply and receives current via two copper terminals 214a,b. The cathode 212a is connected to the ground and exports gases to gas chromatography (GC). The tubes are made of 304 stainless steels, and the internal diameter (ID) and outside diameter (OD) are 3.05 mm and 6.35 mm, respectively. The electrode assembly is enclosed in a quartz tube 216 to control the volume of the experimented gas for research purposes. The quartz tube's ID and OD are 10.49 mm, and 6.35 mm. The whole unit is enclosed by a 304.80 mm long iron tube cage 218 to isolate the electromagnetic field produced at high voltage-short pulses. The volume of gas plasma was controlled for research accuracy purposes with a quartz glass tube. Since pressure build-ups happen at various flow rates, The system was designed to operate at a higher pressure than atmospheric pressure for safety.

### Electrical Discharges

Electricity is used to create gas plasma, and since the ultimate goal is to create a non-thermal plasma rather than preheating the gas mixture, an FPG 30-1NM pulse generator is used to produce nanoseconds discharges. Pulses are required to be in nanoseconds to not let the temperature increase by avoiding continuous electrical flow. Electricity current streams through two copper-made terminals to the tubes. Each pulse width is about 3 ns with a maximum energy input of 3 mJ. The PRF varied between 3 kHz and 10 kHz, and the experimented input voltage range was between 20 kV and 30 kV to investigate the H₂ yield (kg/kJ), methane conversion, and SEI. The input voltages and PRF were changed randomly for each experiment to ignore biased errors. Changing the input voltage or pulse repetition frequency changes the input power and, therefore, the plasma medium characters. In fact, as is discussed in follow, adding power to the gas mixture does not necessarily produce more hydron effectively. Producing high voltage-short pulses will create a strong electric magnetic field. Experimentally, we designed a metal cage which can control the magnetic field. Indeed, by having electric magnetic field around the reformer no sensor can operate so isolating the magnetic field is crucial to be able to have nanosecond pulses for reforming. This study aims to reform methane to hydrogen using a continuous flow process and since pulses are in nanoseconds, measuring the current is a hard job, so a computational model was developed to predict the current through the system.

### Membrane Design

Regardless of which method is used to produce H₂, separation and purification processes are required. Currently, H₂ can be purified through one or a combination of three major processes: 1) pressure swing adsorption (PSA), 2) fractional/cryogenic distillation, or 3) membrane separation. Not only are membrane separations promising to separate hydrogen because of their low energy consumption, the possibility for continuous operation, lower investment cost, ease of operation, and, therefore, cost-effectiveness, they are capable of operating in a non-thermal process, which is in favor of this research.

Methane is being used as the feedstock in this work. After reforming methane through the plasma-based reformer, H₂, C₂H₂, C₂H₆, C₃ₛ, and soot will be produced. Using an efficient membrane separates H₂ from other light hydrocarbons and consumes low energy for producing a kilogram of hydrogen. Membranes are often categorized by their materials: metallic, inorganics, porous carbons, purely organic polymers, and hybrids or composites (2). To evaluate the cost of produced H₂ per kilogram for the plant, the Aspen Plus simulator is used to design the membrane. Moreover, Weller and Steiner's law confirms the model; sensitivity analysis for changing pressure along the membrane is performed (3). 99.76% hydrogen separation from methane is achieved by the designed membrane. 305000 nanotubes in the membrane are required to handle 32000 m3/hr flow. The membrane length is 95 cm, and the diameter of each tube is 1 nanometer. Permeated H₂ and CH₄ in the membrane are calculated by below equations:

$$Permeated {H}_{2} ( kmol / s ) = Rho \times MWC \times J \times ID \times L \times PI ( ) \times NTUBES / MWH$$

$$Permeated {CH}_{4} ( kmol / s ) = CR \times J \times ID \times L \times PI ( ) \times NTUBES / ( CP \times MWC )$$

where Rho is the density of the mixture gas exiting the plasma reformer (kg/m3), MWC and MWH are methane and hydrogen molecular weights (kg/kmol), J is the volumetric flux (m/s), ID is the diameter of tubes (m), L is the length of tubes (m), NTUBES is the number of tubes in the membrane, CR is the methane concentration (kg/m3) in the return stream, and CP is the permeated CH₄ concentration (kg/m3).

Table 1 shows all formulas and their descriptions for modeling the hydrogen membrane separation. Membrane modules consist of many individual hollow fibers that allow flexibility in meeting pressure drop and performance constraints.

TABLE 1
Membrane design model inputs and mathematical calculation
Model Input

| | | |
|---|---|---|
| MWC | 16.04 | kg/kmol methane molar weight |
| MWH | 2.016 | kg/kmol H₂ molar weight |
| MU | 0.00001 | g/cm/s viscosity |
| PMF | CH4_FEED*MWC | methane flow feed stream |
| TMF | TOT_FEED*MW_FEED | the total mass of the mixture flow |

MIX STREAM

| | | |
|---|---|---|
| RHO | DENS_FEED/1000 | g/cm3 density |
| FIN | TOT_FEED*MW_FEED/RHO*3600 | L/hr feed stream flow rate |
| CIN | PMF/TMF*RHO*1000 | g/L feed stream CH4 concentration |
| PIN | PRES_FEED/101325 | atm feed stream pressure |

INTERMEDIATE

| | | |
|---|---|---|
| UAVE | FIN/(PI( )/4*ID\hat{ }2*NTUBES)*10/36 | cm/s bulk average velocity |
| RE | ID*UAVE*RHO/MU | Reynolds number |
| SC | MU/RHO/DIFF | Schmidt number |
| MT_1 | COEF1 | |
| MT_2 | RE\hat{ }COEF2 | |
| MT_3 | SC\hat{ }COEF3 | |
| MT_4 | (ID/LEN)\hat{ }COEF4 | |

RESULTS

| | | |
|---|---|---|
| CP | GEL_CONC*(1-REJ_COEF) | g/L permeated CH4 concentration |
| MT | MT_1*MT_2*MT_3*MT_4 | coefficient |
| K | MT*DIFF/DIAM/100 | m/s mass transfer coefficient |
| J | K*LN((GEL_CONC-CP)/(CIN-CP)) | m/s volumetric flux |
| FP | J*ID*L*PI( )*NTUBES*100*36/10 | L/h permeated stream flow rate |
| PR | (PIN-DELTA_P)*101325 | N/m2 return stream pressure |
| CR | (CIN-FP/FIN*CP)/(1-FP/FIN) | g/L return stream CH4 concentration |

Table 2 shows the results for a scenario assuming 32000 m3/hr (8.89 m3/s) flow of 70% CH₄ and 30% H₂ molar concentration from the plasma reformer. The pressure in the membrane drops by a factor of 9.9. The permeate and retentate streams' pressures are 10132.5 and 100312.0 N/m2, i.e., the pressure on the permeate side is higher by a factor of 9.9 compared to the retentate side to reach 99.76% H₂ purity. The sensitivity analysis for pressure changes is included in the next discussion section. The results are presented in Table 3, which shows that 99.21% of total generated H₂ by plasma reformer can be separated along the membrane. Based on experimental data the produced molar concentration of hydrogen in the plasma reformer does not exceed 34%.

TABLE 2
Membrane design results assuming 8.89 m3/s mixed gas feeding
with 30% molar concentration of hydrogen.

| OUTPUT | RETENTATE | PERMEATE | MIX |
|---|---|---|---|
| H2 (kmol/s) | 0.00086688 | 0.109370338 | 0.110237 |
| CH4 (kmol/s) | 0.25315376 | 0.000267871 | 0.253422 |
| Total flow | 0.25402064 | 0.10963821 | 0.363659 |
| (kmol/s) | | | |
| Temp (K) | 298.15 | 298.15 | 298.15 |
| Pressure | 100311.75 | 10132.5 | 1011325 |
| (N/m²) | | | |
| H2 (kg/s) | 0.00174753 | 0.220477 | 0.222225 |
| CH4 (kg/s) | 4.06128 | 0.00429739 | 4.06558 |
| Total flow | 4.06303 | 0.224775 | 4.28781 |
| (kg/s) | | | |

Using the complete-mixing model for gas separation by membranes, if there is no sweep gas and both sides of the membrane are well-mixed, the flux analysis can be calculated using steady-state mass balances. When the permeated flow rate is a small fraction of the entering feed rate, there is a minimal change in composition. Then the results derived using the complete-mixed model deliver satisfactory estimates of permeate purity. This is driven by Weller and Steiner. The mass flow of H₂ out of the permeate side is calculated by multiplying the mole fraction of H₂ on permeate side (Yₚ) by (Vₚ) permeate flow-out rate. Pₕ is the pressure on the retentate side, and Pₗ is the pressure on the permeate side. P_{A} is the permeability of H₂ in the membrane, Aₘ and T are the membrane area and thickness, PₕX₀ is the partial pressure of H₂ on the rejected side, and PₗYₚ is the partial pressure on the permeate side.

Yp is calculated as follows:

$${V}_{p} \times {Y}_{p} = ( {P}_{A} / T ) \times ( {P}_{h} \times {X}_{0} - {P}_{l} \times {Y}_{p} ) \times {A}_{m}$$

$${Y}_{p} = ( - b + \sqrt{( {b}^{2} - 4 a c )} ) / 2 a$$

$$a = 1 - {α}_{H 2 / C H 4}$$

$$b = - 1 - {α}_{H 2 / C H 4} + ( 1 / r ) + ( {X}_{0} / r ) ( {α}_{H 2 / C H 4} - 1 )$$

$$c = {α}_{H 2 / C H 4} / r$$

$$r = {P}_{l} / {P}_{h}$$

The ideal selectivity (α_{CH2/CH4}) is calculated by P_{A}/P_{B}, in which P_{B} is the permeability of methane in the membrane. α_{CH2/CH4} is assumed to be 101.93, and pressure on the retentate side is higher than permeate side by a factor of 9.9 as for the membrane that is designed by Aspen Plus.

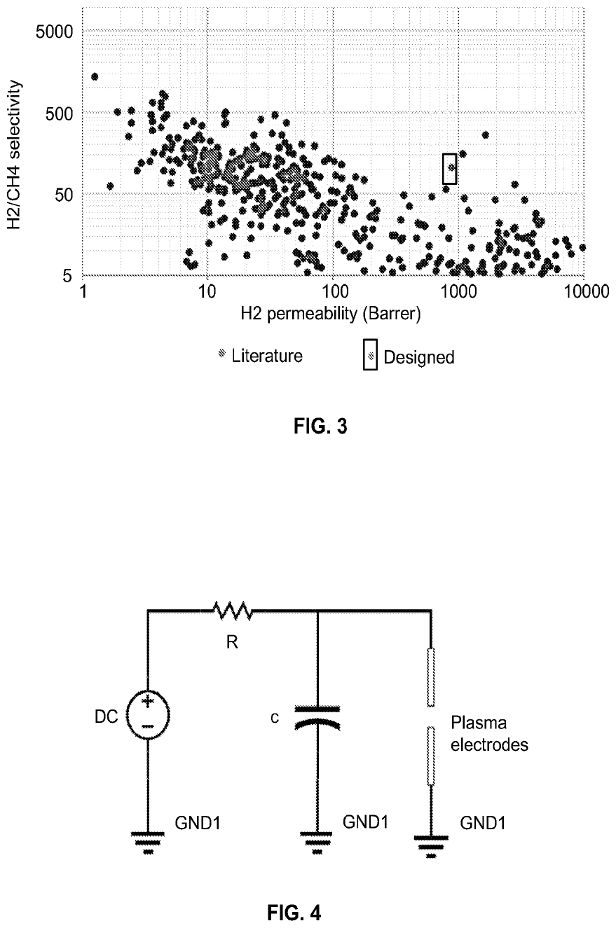

FIG. 3 shows experimental data for α_{CH2/CH4} extracted from other developed researches and the designed membrane in this research. FIG. 4 explains that as the H₂ permeability increases, the selectivity of H₂ to CH₄ (ideal selectivity) decreases.

### Example 2

### Methodology

CH₄ is converted to H₂ through a nanosecond repetitive pulse plasma discharge. When a high voltage pulse is applied across electrons, electrons and ions are accelerated. As enough energy is implied, electrons gain enough energy to break through and flow between two electrodes. While electrons move from anode to cathode, electrons and the ions collide with other neutral molecules in the reactor and create more electrons and ions.

As a consequence of these impacts and reactions, an arc or a glow will form. The default settings for power source parameters are the pulse rise and fall time (each=1 ns) and the pulse width (=2 ns, apart from the rise and fall time). All parameters were fixed at each experiment except one parameter to study the effect of energy input, pulse repetition frequency, and feedstock flow rate on the methane conversion and H₂ production. Table 3 represents the conditions of the experiments.

TABLE 3
Experimental variables for the nanosecond plasma discharge.

| Parameter | Input voltage (kV) | PRF (kHz) | Flow rate (L/min) |
|---|---|---|---|
| Values | 22, 24, 26, 28, 30 | 4, 5, 6, 7, 8, 9, 10 | 1, 2 |

Two gas detectors are used in GC for detecting hydrogen and co-products. In addition to a GC-HID (column Seive13) detector for detecting H₂, a GC-TCD (Hypes D) detector is used to measure the mass fraction of C₂, C₃, C₄, C₅, and CH₄. Each experiment is repeated four times at random hours. The maximum accepted error for the GC-gas analysis is less than 5%. To reduce unwanted GC-related errors, the GC was in an operation mode for the entirety of the experiment. Helium gas was chosen as the career gas for the GC at 21 kPa pressure. The GC was mini-cooked for 10 minutes at 220° C. after each experiment. Gas chromatography operation control sequences and setups are summarized in Tables 4A and 4B. For the temperature sequence control two columns were used: HayeSep D and molecular sieve 13X.

TABLE 4A
The temperature sequence control for GC columns

| Initial | Hold | Ramp | Final |
|---|---|---|---|
| temperature (° C.) | (min) | (deg/min) | temperature (° C.) |
| 50.00 | 6.00 | 40.00 | 200.00 |
| 200.00 | 10.00 | 20.00 | 200.00 |

TABLE 4B
GC-valves control to feed the reformed gases into columns

| Time (min) | Event |
|---|---|
| 0.00 | Zero |
| 0.00 | Sound |
| 0.10 | G ON (Valve rotate) |
| 1.50 | A ON (Stop Flow) |
| 4.00 | A OFF (Stop Flow) |
| 4.00 | G OFF (Valve rotate) |

### Gas Delivery and Analysis

Pure methane was used as the reactant feedstock to study the CH₄ conversion (mass percentage) and H₂ yield (kg/kJ) at various energy inputs, flow rates, and PRF. The gas was injected at a continuous flow of 1.1 L/min and 2.2 L/min. Two calibrated mass flow controllers (Alicat) measured gas temperature, pressure, and mass flow rate at the input and output of the reformer. The discharged products were analyzed with a calibrated GC-SRI 8610C equipped with a thermal conductivity detector (TCD) and a helium ionized detector (HID).

The methane is fed to the reactor with a flow rate of Q. Voltage and PRF are applied to the anode tube, which delivers gas into the reformer at a continuous flow. As the gas travels between two electrodes, reactions will take place. The reformed (mixture) gas exits the reformer from the cathode tube toward GC. GC simultaneously analyzes the produced gases.

### Products Analysis

It has been demonstrated that increasing the voltage or the repetition frequency suppresses carbon formation. From experimental observations, in addition to the gaseous products, the inner wall of the quartz tube and the surface of the central electrodes were coated with carbon black. Accordingly, solid carbon production was ignored in evaluating CH₄ conversion.

The amount of a few co-products was too small to allow confirmation of specific components; they were categorized by the number of their carbons, i.e., C₂H₂, C₂H₄, C₂H₆, and C₃. Product analysis considers the CH₄ conversion, H₂ selectivity, SEI (kJ/kg), and H₂ yield (kg/kJ). The methane conversion (x_{CH}_{₄}(%)) and SEI (kJ/kg) are calculated as follows:

$${x}_{{CH}_{4}} = ( mole of {CH}_{4} consumed / moles of {CH}_{4} introduced ) \times 100 \% \qquad Eq . 1$$

$$SEI = \frac{P}{{Q}_{in} \times ρ} \qquad Eq . 2$$

where P is the input power (W), ρ is density (kg/m³), and Qᵢₙ is the inflow rate (m³/s). The yield of hydrogen (Y_{H}_{₂}) and selectivity (Sᵢ) are calculated as follows:

$${Y}_{{H}_{2}} = \frac{{x}_{{H}_{2}} \times {MW}_{{H}_{2}}}{M {W}_{i n} \times SEI} \qquad Eq . 3$$

$${Y}_{{C}_{x} {H}_{y}} ( \% ) = \frac{{x}_{{C}_{x} {H}_{y}} \times M {W}_{{C}_{x} {H}_{y}}}{M {W}_{i n} \times SEI} \times 100 \qquad Eq . 4$$

$$\text{?} \qquad Eq . 5$$

$$\text{?} \text{indicates text missing or illegible when filed}$$

in which x (x=2, 3) and y respectively represent the number of carbon and hydrogen atoms in hydrocarbon, MW is the molecular weight, and Y_{H}_{₂} is in kg/kJ.

### Example 3

### Predicted Reactions: Simulating Plasma-Based Reformer

The reformer is simulated in a one-dimensional hybrid model. Since including all the chemical reactions of the particles in the plasma is an original and very laborious task, it is challenging to develop an exactly precise, fully understood model. This model treats electrons' energy (temperature) kinetically, using the Maxwell-Boltzmann equation. Electron-induced vibration, electron-induced ionization, and elastic impact between electron and hydrocarbon molecules are considered. Ions and neutrals are solved with fluid dynamic momentum and energy conservation equations for a multi-component mixture. Continuity equations are solved for each mixture component as shown.

The electron density and mean electron energy are computed by solving a pair of drift-diffusion equations. The convention of electrons due to fluid motion is ignored. The electron density continuity equation defines the electron conservation:

$$\frac{\partial {n}_{e}}{\partial t} + ∇ \cdot \vec{{Γ}_{e}} = {\dot{n}}_{e} \qquad Eq . 6$$

$${\dot{n}}_{e} = {\sum }_{j = 1}^{n} {χ}_{j} {α}_{j} {N}_{n} \vert {Γ}_{e} \vert \qquad Eq . 7$$

where \vec{Γ}ₑ (Eq. 8) is the electron density flux, and \dot{n}ₑ is the source of electrons produced or consumed during ionization or attachments, αⱼ is the Townsend coefficient for reaction j.

$$\vec{{Γ}_{e}} = {μ}_{e} {n}_{e} ∇ ϕ - {D}_{e} ∇ {n}_{e} \qquad Eq . 8$$

where φ is the electric potential, μₑ is the electron mobility and is the Dₑ the electrons diffusion coefficient and is calculated by the Boltzmann equation. The electron energy loss is obtained by summing the collisional energy loss for all reactions:

$${\dot{n}}_{ε} = {\sum }_{1}^{p} {χ}_{j} {k}_{k} {N}_{n} {n}_{e} Δ {ε}_{j} \qquad Eq . 9$$

$$\frac{\partial {n}_{ε}}{\partial t} + ∇ \cdot \vec{{Γ}_{ε}} + ∇ ϕ \cdot \vec{{Γ}_{ε}} = {\dot{n}}_{ε} \qquad Eq . 10$$

$$\vec{{Γ}_{ε}} = {μ}_{ε} {n}_{ε} ∇ ϕ - {D}_{ε} ∇ {n}_{ε} \qquad Eq . 11$$

where Δεⱼ is the energy loss from reaction j. The rate coefficient, kₖ, is computed from cross-section experimental data:

$${k}_{k} = γ {\int }_{0}^{∞} ε {σ}_{k} ( ε ) f ( ε ) d ε \qquad Eq . 12$$

$$γ = \sqrt{\frac{2 q}{{m}_{e}}} \qquad Eq . 13$$

where mₑ is the electron mass, ε is energy in volt, σₖ is collision cross-section, and ƒ(ε) is the electron distribution function which in this case, look up tables for the mobility and mobility energy are uploaded from previous empirical data. For positive ions in the discharge, the conservation equation is given by:

$$\frac{\partial {n}_{i}^{+}}{\partial t} + ∇ \cdot ( {n}_{i}^{+} \vec{u} ) = - ∇ {\vec{{Γ}_{ι}}}^{+} + {n}_{i}^{+} where {n}_{i}^{+} \qquad Eq . 14$$

is the density of positive ions, \vec{u} is the mass average fluid velocity, and \vec{Γ}_{τ}⁺ is the mass flux of ion spices i relative to the mass average velocity u. For the average mass density, the continuity equation is written as:

$$\frac{\partial n}{\partial t} + ∇ \cdot ( n \vec{u} ) = 0 \qquad Eq . 15$$

where n=Σnₖ is the total mass density of the fluid (positive ions, electronically excited atoms, and neutrals).

Several formulations are available for modeling mass transport, including convection, diffusion equation, and Maxwell-Stefan equations. Since computing Maxwell-Stefan for more than six spices is expensive, a mixture-average diffusion model is assumed, i.e., the model assumes that the relative mass flux due to molecular diffusion is governed by Fick's law type approximation. Therefore, for non-electron species, the following equation is solved for the mass fraction of each species.

$$ρ \frac{\partial }{\partial t} ( {w}_{k} ) + ρ ( u \cdot ∇ ) {w}_{k} = ∇ \cdot {j}_{k} + {R}_{k} \qquad Eq . 16$$

wₖ is the mass fraction of kth species, jₖ is the diffusive flux vector, and Rₖ is the rate expression for species k. jₖ for mixture-average diffusion and Rₖ are calculated based on Arrhenius law as:

$${j}_{k} = ρ {w}_{k} {V}_{k} \qquad Eq . 17$$

$${V}_{k} = {D}_{k , m} \frac{{∇}_{{w}_{k}}}{{w}_{k}} + {D}_{k , m} \frac{∇ {M}_{n}}{{μ}_{n}} + {D}_{k}^{T} \frac{∇ T}{T} - {z}_{k} {μ}_{k , m} ∇ \cdot ϕ \qquad Eq . 18$$

$${D}_{k , m} = \frac{1 - {w}_{k}}{{\sum }_{k = 1}^{s} {x}_{j} / {D}_{K j}} \qquad Eq . 19$$

$${R}_{k} = {M}_{k} {\sum }_{j = 1}^{N} {v}_{{k}_{j}} {r}_{j} \qquad Eq . 20$$

$${r}_{j} = {k}_{f , j} {\prod }_{k = 1}^{s} {c}_{k}^{{v}_{kj}^{′}} - {k}_{r . j} {π}_{k = 1}^{s} {c}_{k}^{{v}_{kj}^{″}} \qquad Eq . 21$$

$${K}_{j} = {A}_{j} {T}^{{β}_{j}} exp ( - \frac{{E}_{j}}{R T} ) \qquad Eq . 22$$

where Vₖ is the multi-component diffusion for species k, D_{k,m} is the mixture average diffusion coefficient, which is based on Maxwell-Stefan equations, where isobaric and isothermal conditions have been assumed. My is the mean molar mass of the mixture, T is the gas temperature,

$${D}_{k}^{T}$$

is the thermal diffusion coefficient for species k. zₖ is the charge number for species k, k_{f} is the forward rate of reaction for jₜₕ reaction,

$${v}_{kj}^{″}$$

is the stoichiometric matrix corresponding to forwarding reaction,

$${v}_{k j}^{″}$$

is the sociometric matrix corresponding to reverse reactions, Aⱼ is the frequency factor for reaction j, βⱼ is the temperature exponent and E is the activation energy for reaction j, and μ_{k,m} is the mixture average mobility for species k, which is calculated by Einstein's relation:

$${μ}_{k , m} = \frac{q {D}_{k , m}}{{k}_{B} T} \qquad Eq . 23$$

where q is the unit charge, k_{B} is Boltzmann constant.

A modified local field approximation using Bolsig+ was employed for obtaining electron transport mobility and diffusion coefficients. The mobility and diffusion coefficients from other experiments were utilized in the model to obtain electric field properties. A mixed average model was applied for the diffusion model.

Twenty fundamental particles and eighty-three main reactions are considered. The main reactions considered in the simulation model and their corresponding references are listed in Table 5. The number of reactions has been simplified to improve computational efficiency.

TABLE 5
Main reactions utilized in the simulation model, reaction rate
coefficients and the corresponding reference

| Reaction | | Reaction rate |
|---|---|---|
| No. | Reaction equation | coefficient |
| 1 | CH₄ + e → CH₄(ν = 1) + e | Bolsig+ |
| 2 | CH₄ + e → CH4(ν = 2) + e | Bolsig+ |
| 3 | CH₄ + e → CH₄(ν = 3) + e | Bolsig+ |
| 4 | C₂H₂ + e → C₂H₂(ν = 1) + e | Bolsig+ |
| 5 | C₂H₂ + e → C₂H₂(ν = 2) + e | Bolsig+ |
| 6 | C₂H₂ + e → C₂H₂(ν = 3) + e | Bolsig+ |
| 7 | C₂H₄ + e → C₂H₄(ν = 1) + e | Bolsig+ |
| 8 | C₂H₄ + e → C₂H₄(ν = 2) + e | Bolsig+ |
| 9 | C₂H₆ + e → C₂H₆(ν = 1) + e | Bolsig+ |
| 10 | C₂H₆ + e → C₂H₆(ν = 2) + e | Bolsig+ |
| 11 | C₂H₆ + e → C₂H₆(ν = 3) + e | Bolsig+ |
| 12 | H₂ + e → H₂⁺ + 2e | Bolsig+ |
| 13 | e + CH₄ → CH + 3H + e | Bolsig+ |
| 14 | e + CH₄ → C + 4H + e | Bolsig+ |
| 15 | e + CH₃ → CH₂ + H + e | Bolsig+ |
| 16 | e + CH₃ → CH + 2H + e | Bolsig+ |
| 17 | e + CH₂ → CH + H + e | Bolsig+ |
| 18 | e + CH₂ → C + 2H + e | Bolsig+ |
| 19 | e + CH → C + H + e | Bolsig+ |
| 20 | e + C₂H₄ → C₂H₂ + 2H + e | Bolsig+ |
| 21 | e + CH₃ → CH₃⁺ + 2e | Bolsig+ |
| 22 | e + CH₃ → CH₂⁺ + H + 2e | Bolsig+ |
| 23 | e + CH₂ → CH₂⁺ + 2e | Bolsig+ |
| 24 | e + CH₂→ CH⁺ + H + 2e | Bolsig+ |
| 25 | e + CH → CH⁺ + 2e | Bolsig+ |
| 26 | e + CH → CH⁺ + 2e | Bolsig+ |
| 27 | e + C₂H₂ → C₂H₂⁺ + 2e | Bolsig+ |
| 28 | e + C₂H₄ → C₂H₂⁺ + 2e + H₂ | Bolsig+ |
| 29 | e + C₂H₄⁺ → C₂H₂ + 2H | 2.00 × 10⁻³³ |
| 30 | C⁺ + CH₄ → C₂H₂⁺ + H₂ | 4.00 × 10⁻¹⁶ |
| 31 | C⁺ + CH₃ → C₂H₂⁺ + H | 1.30 × 10⁻¹⁵ |
| 32 | C⁺ + CH₂ → CH₂⁺ + C | 5.20 × 10⁻¹⁶ |
| 33 | C⁺ + CH → CH⁺ + C | 3.80 × 10⁻¹⁶ |
| 34 | C⁺ + C₂H₄→ C₂H₄⁺ + C | 1.70 × 10⁻¹⁷ |
| 35 | C₂H₂⁺ + C₂H₄→ C₂H₄⁺ + C₂H₂ | 4.14 × 10⁻¹⁶ |
| 36 | CH⁺ + CH₄ → C₂H₄⁺ + H | 6.50 × 10⁻¹⁷ |
| 37 | CH⁺ + CH₄ → C₂H₂⁺ + H₂ + H | 1.43 × 10⁻¹⁶ |
| 38 | CH⁺ + H₂ → CH₂⁺ + H | 1.20 × 10⁻¹⁵ |
| 39 | CH⁺ + H → C⁺ + H₂ | 7.50 × 10⁻¹⁶ |
| 40 | CH₂⁺ + CH₄ → C₂H₂⁺ + 2H₂ | 3.97 × 10⁻¹⁶ |
| 41 | CH₂⁺ + CH₄ → C₂H₄⁺ + H₂ | 8.40 × 10⁻¹⁶ |
| 42 | CH₂⁺ + H₂ → CH₃⁺ + H | 1.60 × 10⁻¹⁵ |
| 43 | CH₂⁺ + CH₄ → CH₃⁺ + CH₃ | 1.38 × 10⁻¹⁶ |
| 44 | CH₃⁺ + CH₄ → CH₄⁺ + CH₃ | 1.36 × 10⁻¹⁶ |
| 45 | CH₃⁺ + CH → C₂H₂⁺ + H₂ | 7.10 × 10⁻¹⁶ |
| 46 | CH₄⁺ + C₂H₄ → C₂H₄⁺ + CH₄ | 1.38 × 10⁻¹⁶ |
| 47 | CH₄⁺ + C₂H₂ → C₂H₂⁺ + CH₄ | 1.13 × 10⁻¹⁵ |
| 48 | H⁺ + CH₄ → CH₄⁺ + H | 1.50 × 10⁻¹⁷ |
| 49 | H⁺ + CH₄ → CH₃⁺ + H₂ | 2.30 × 10⁻¹⁷ |
| 50 | H⁺ + CH₃ → CH₃⁺ + H | 1.40 × 10⁻¹⁵ |
| 51 | H⁺ + CH₂ → CH₂⁺ + H | 1.40 × 10⁻¹⁵ |
| 52 | H⁺ + CH₂ → CH⁺ + H₂ | 1.90 × 10⁻¹⁵ |
| 53 | H⁺ + CH → CH⁺ + H | 1.90 × 10⁻¹⁵ |
| 54 | H⁺ + C₂H₄ → C₂H₄⁺ + H | 1.00 × 10⁻¹⁵ |
| 55 | H⁺ + C₂H₄ → C₂H₂⁺ + H₂ + H | 1.00 × 10⁻¹⁵ |
| 56 | H⁺ + C₂H₂ → C₂H₂⁺ + H | 5.40 × 10⁻¹⁶ |
| 57 | H₂⁺ + CH₄ → CH₄⁺ + H₂ | 1.40 × 10⁻¹⁵ |
| 58 | H₂⁺ + CH₄ → CH₃⁺ + H₂ + H | 2.30 × 10⁻¹⁵ |
| 59 | H₂⁺ + CH₂ → CH₃⁺ + H | 10.00 × 10⁻¹⁶ |
| 60 | H₂⁺ + CH₂ → CH₂⁺ + H₂ | 10.00 × 10⁻¹⁶ |
| 61 | H₂⁺ + CH → CH₂⁺ + H | 7.10 × 10⁻¹⁶ |
| 62 | H₂⁺ + CH → CH⁺ + H₂ | 7.10 × 10⁻¹⁶ |
| 63 | H₂⁺ + C → CH⁺ + H | 2.40 × 10⁻¹⁵ |
| 64 | H₂⁺ + C₂H₄ → C₂H₄⁺ + H₂ | 2.21 × 10⁻¹⁵ |
| 65 | H₂⁺ + C₂H₄ → C₂H₂⁺ + 2H₂ | 8.82 × 10⁻¹⁶ |
| 66 | H₂⁺ + C₂H₂ → C₂H₂⁺ + H₂ | 4.82 × 10⁻¹⁵ |
| 67 | H₂⁺ + H → H⁺ + H₂ | 6.40 × 10⁻¹⁶ |
| 68 | CH + CH → C₂H₂ | 2.00 × 10⁻¹⁶ |
| 69 | CH + CH₂ → C₂H₂ + H | 6.60 × 10⁻¹⁷ |
| 70 | CH + CH₄ → C₂H₄ + H | 1.00 × 10⁻¹⁶ |
| 71 | CH + H → C + H₂ | 4.98 × 10⁻¹⁷ |
| 72 | H₂ + CH → CH₂ + H | 3.03 × 10⁻¹⁷ |
| 73 | CH₂ + CH₂ → C₂H₂ + H₂ | Arrhenius |
| | | expression |
| 74 | CH₂ + CH₂ → C₂H₂ + 2H | 5.27 × 10⁻¹⁷ |
| 75 | CH₂ + CH₂ → C₂H₄ | 1.70 × 10⁻¹⁸ |
| 76 | CH₃ + CH₂ → C₂H₄ + H | 7.00 × 10⁻¹⁷ |
| 77 | CH₃ + CH₃ → C₂H₄ + H₂ | 1.7e14 × |
| | | exp(−16000/Tg) |
| 78 | C + CH₄ → C₂H₂ + H₂ | 1.00 × 10⁻¹⁷ |
| 79 | H + CH₂ → CH + H₂ | Arrhenius |
| | | expression |
| 80 | H + CH₃ → CH₂ + H₂ | Arrhenius |
| | | expression |
| 81 | H + CH₃ → CH₄ | 7.00 × 10⁻¹⁸ |
| 82 | H + CH₄ → CH₃ + H₂ | Arrhenius |
| | | expression |
| 83 | H + H + H₂ → H₂ + H₂ | Arrhenius |
| | | expression |

### Electric Circuit

An external electric circuit was modeled to generate plasma in the reactor (FIG. 4). The external circuit contains resistance R, capacitance C, and two electrodes. The inter-electrode distance (d) is fixed at 6 mm, and the capacitance is parallel to the plasma discharge. The input voltage, V_{Pw}, varies from 22 kV to 30 kV to find the breakdown voltage at atmospheric pressure and evaluate the effect of voltage on methane conversion and hydrogen yield. C is fixed at 6.66 pF, corresponding to the estimated value from the power supply. The input energy (IE) and power (p) can be calculated as:

$$IE = \frac{1}{2} C \cdot {V}_{P w}^{2} \qquad Eq . 24$$

$$p = IE \times PRF \qquad Eq . 25$$

This model handles the equations, boundary conditions, and initial conditions relating to electrostatics and charge conservation by solving Gauss and Maxwell's equations. The electrostatics model is solved using a multi-frontal massively parallel direct solver (MUMPS) with an error tolerance of 0.001. The discharge physics model equations consider the temporal and spatial effects perpendicular to electrodes. Based on the local-field approximation, the electron energy (Tₑ) is considered to be a function of the electron density nₑ, and the electric field, E.

### Boundary Conditions

### Electron Density (nₑ)

Since a high voltage nanosecond DC pulse power supply is being used, secondary electron emission exists by the cathode. An electron is emitted from the cathode surface with a specific probability when stuck by an ion. When they acquire enough energy to initiate ionization, these electrons are moved by a strong field close to the cathode. This model assumes electrons are lost to the wall, and there is no reflection. The secondary emission coefficient is assumed to be 0.45 for stainless steel tubes. Heavy species and ions are lost to the wall due to surface reactions and the fact that the electric field is perpendicular to the wall.

### Electric Potential

The electric potential at the cathode is set to zero. The potential at the driven electrode is connected to the external electric circuit. Eq. 13 was solved by the circuit solver for the boundary voltage as follows:

$$\frac{d {V}_{d}}{d t} + \frac{1}{C} ( {I}_{d} - \frac{{V}_{P w}}{R} ) = 0 \qquad Eq . 26$$

where I_{d} is discharge current and V_{d} is the voltage across the boundary.

### Mass Average Velocity (u)

No-slip boundary conditions are set at anode and cathode. Zero gradient boundary condition is maintained.

### Gas Temperature

All electrode boundaries are set to be isothermal walls at room temperature, and zero gradient boundary condition is maintained.

### Numerical Scheme

The numerical scheme for solving the governing equations is based on the finite element approach in a quadratic shape function similar to the experimental results. Reduced electric fields are used to compute electron transportation properties and electron impact rate coefficient for a given reduced electric field. Lookup tables of electrons (eV) versus collision cross-sections (m²) from previous empirical results using Bolsig+ are implied to calculate electron transportation and source coefficients for each electron impact reaction. The equations for electrons, ions, and meta-stable densities are solved by the continuity equations, i.e., density and continuity equation, momentum and Navier Stokes fluid momentum, and energy and energy balance equations. COMSOL is used to generate a mesh and accomplish numerical calculations. The maximum mesh element size is 0.06 mm with 1927 degrees of freedom. The degree of freedom is approximately equal to the number of the products and their dependent variables. The initial densities of neutral particles (np), positive ions (pp), and electrons density (ed) are set as 2.45×10²³ (1/m³), 1×10⁻¹² (1/m³), and 1×10¹⁶ (1/m³), respectively. Gas temperature is 300 K at 101.325 kPa pressure.

$$np = \frac{{P}_{0}}{{k}_{B} \times {T}_{g}} . \qquad Eq . 27$$

where is k_{B} Boltzmann constant (m²·kg/s²/K), T_{g} is the gas temperature (K) and P₀ is the gas pressure (Pa).

### Example 4

### Pulse Parameters

### Input Voltage

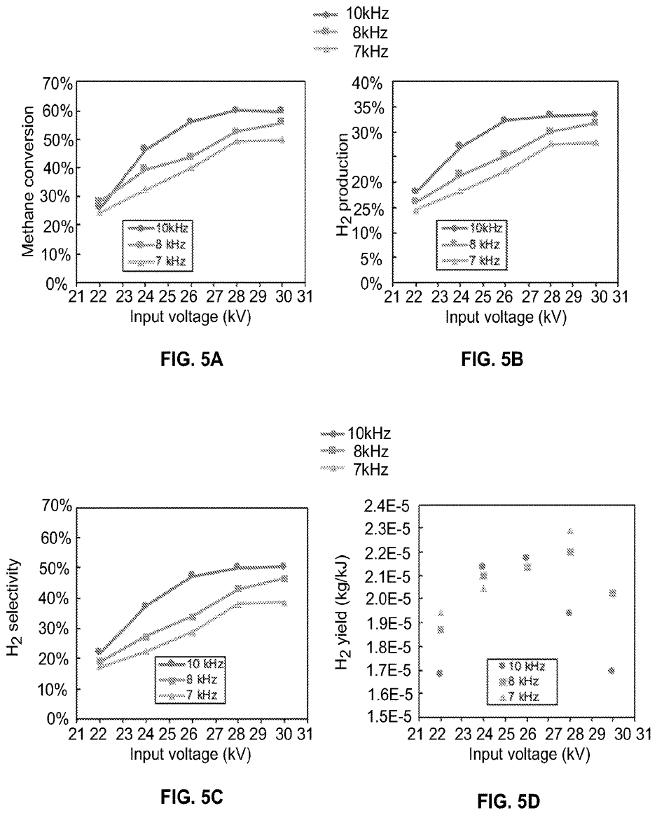

To study the effects of the input voltages on CH₄ conversion and produced H₂, pulse repetition frequency and flow rate are fixed at 10 KHz and 1.1 L/min, respectively. The voltage is varied between 22 kV and 30 kV. Each experiment was repeated four times in random sequences. As shown in FIGS. 5A-5D, increasing the applied voltage enhances CH₄ conversion. Augmenting the applied voltage forms a stronger electric field and increases electrons' energy and density in the discharge zone. Therefore, the probability of gas molecules' collisions and electron impact reactions can be higher. Moreover, rising the input voltage will increase the number of reactive species, and as the number of reactive species in the plasma zone grows, the CH₄ conversion will also enhance. 60.01% methane conversion was achieved at a 30 kV input voltage and 8 kHz of PRF, followed by 59.55% at a 30 kV input voltage and 10 KHz of PRF.

As shown in FIGS. 5A-5D, increasing input voltage and frequency generally enhances CH₄ conversion. The minimum and maximum percentage of CH₄ conversion is observed at 22 kV and 30 kV input voltage at any applied PRF. For instance, augmenting the input voltage from 22 kV to 28 KV increases the CH₄ conversion from 26.05% to 59.62% at 10 KHz. At 8 kHz, methane conversion increases from 27.39% to 55.76% by changing voltage from 22 kV to 30 kV.

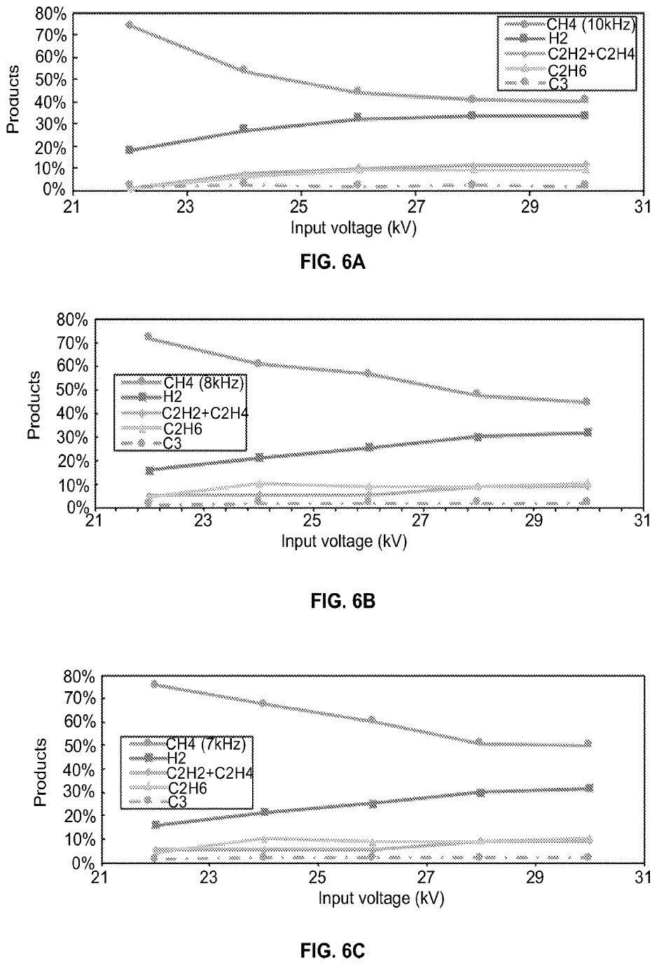

Increasing applied voltage increases H₂ production directly. Observations show that the maximum H₂ production, 36.6%, is at 30 kV and 10 KHz. At 10 KHz, the H₂ production is 36.23%, 33.29%, 32.20%, 30.05%, and 17.95% at 30 kV, 28 kV, 26 kV, 24 kV, and 22 kV, respectively (FIGS. 6A-6C). As the numbers demonstrate, the H₂ production is reduced by decreasing input voltage, proving the direct effect of the input voltage on H₂ production. Observation shows that at the maximum applied voltage and PRF, i.e., 30 kV and 10 kHz, hydrocarbons with two carbon atoms, such as C₂H₂ and C₂H₄, produce more. The production of higher hydrocarbons and their sensitivity to voltage and frequency is much less than hydrogen production herein.

### Pulse Frequency Repetition

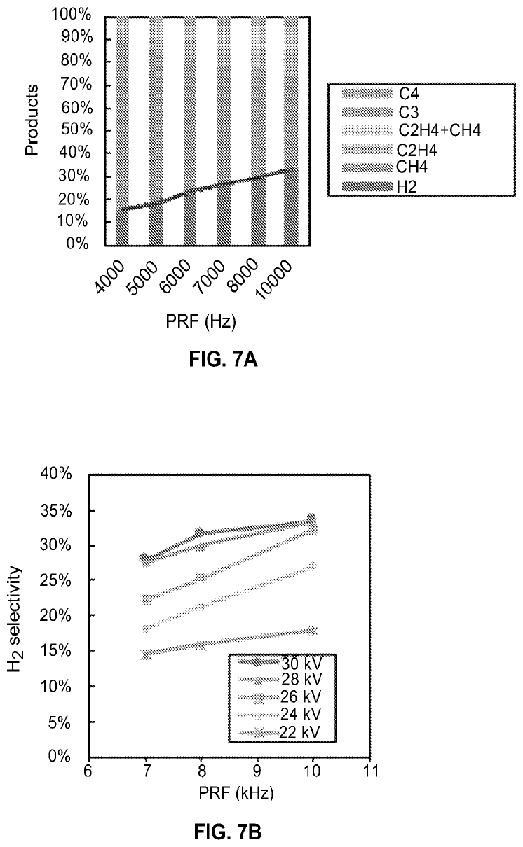

The input voltage and flow rate were stabled at 28 kV and 1.1 L/min to study the effect of pulse repetition frequency on the conversion of CH₄ and produced H₂ (FIGS. 7A-7B) The PRF is the number of pulses in one second and was varied between 4 and 10 KHz. As shown in FIGS. 7A-7B, increasing frequency generally increases CH₄ conversion. Higher pulse frequency can result in less ascent and descent time (54). In fact, the relative species formed in the former pulse discharge enhance the subsequent pulse discharge more effectively. At a constant input energy (=28 kV), the PRF was increased between 4 kHz and 10 KHz to observe the hydrogen production and methane conversion. As FIGS. 7A-7B show, the H₂ linear trend is y (%)=0.0036x+0.1192 with R-squared value of 0.9825. increasing PRF from 4 kHz to 10 kHz, the methane conversion raised from 26.31% to 59.16%, and hydrogen production increased from 15.80% to 33.31%, more than double. Moreover, as the PRF and input energy increase, the produced co-products grow. The acetylene and ethylene production increased from 3.50% to 11.28% by increasing the PRF from 4 kHz to 10 KHz at a constant input voltage (=28 kV).

Although higher CH₄ conversion can be achieved by a higher PFR and input voltage, SEI should be considered to compare the amount of converted CH₄ and produced H₂ per unit of energy for different input voltages and pulse repetition frequencies. Results from GC presented in FIGS. 7A-7B showed that increasing pulse repetition frequency has less effect on methane conversion than the input voltage. In fact, changing the input voltage by 2 kV has more effect on changing power than increasing the PRF by 2 kHz (Eq. 25). For instance, with the input voltage of 30 kV and PRF 10 KHz, the power is 30 W. The input power is 26.13 (W) when the input voltage is 28 kV, and PRF is 10 KHz. At a 30 KV input voltage and 8 KHz PRF the power is 24.00 (W). Hence, we prefer high input voltages and fewer pulses in one second to reduce input power while enough power is applied to the system.

### Power and Specific Energy Input

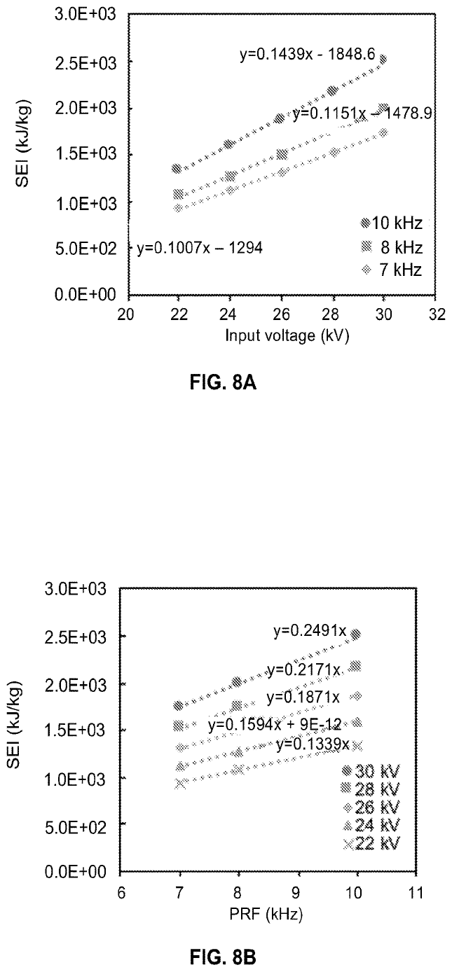

As shown in FIGS. 8A-8B, SEI linearly relates to the input energy and PRF. It also shows that PRF has a linear relation with SEI. Changing PRF increases the specific energy input at a higher rate in comparison to the input voltage. For instance, the SEI for a 30 kV input voltage and 8 kHz of PRF is 25% lower than the SEI at a 30 KV input voltage and 10 KHz of PRF. The SEI is 14% lower at the 28 kV input voltage and 10 KHz of PRF compared to a 30 KV of input voltage at 10 KHz PRF. Since PRF has a lower effect on producing H₂, lower PFR is preferred to reach the maximum H₂ yield (kg/kJ).

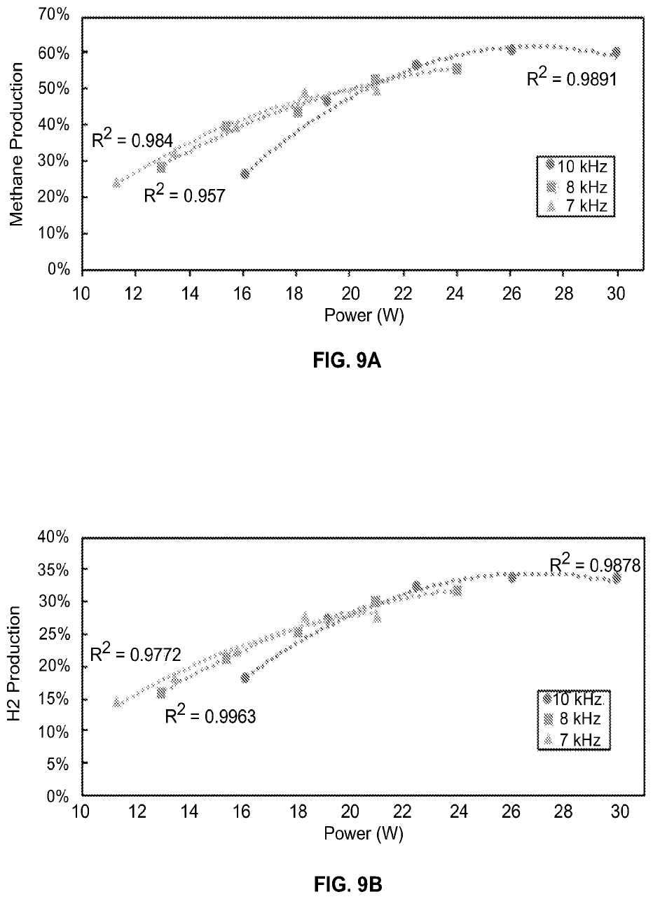

According to Eq. 2, SEI depends on input power and flow-in rate. When the flow was stabled at 1.1 L/min, SEI was only dependent on the power. FIGS. 9A-9B show the effect of power (W) on methane conversion and hydrogen production at a 1.1 L/min (stabled) input flow. Generally, increasing power enhanced CH₄ conversion and H₂ production. The methane conversion at a 26.13 W input power, 28 kV input voltage, and 10 KHz of PRF is 60.1%, which is the maximum methane conversion achieved (FIGS. 8A-8B). The maximum hydrogen production (=34.89%) was achieved at a 30 W input power, 30 kV input voltage, and 10 KHz of the PRF (FIGS. 9A-9B). It is also illustrated that increasing the input energy above 26.13 W does not substantially affect converting CH₄ and H₂ production when the flow is stabled at 1.1 L/min.

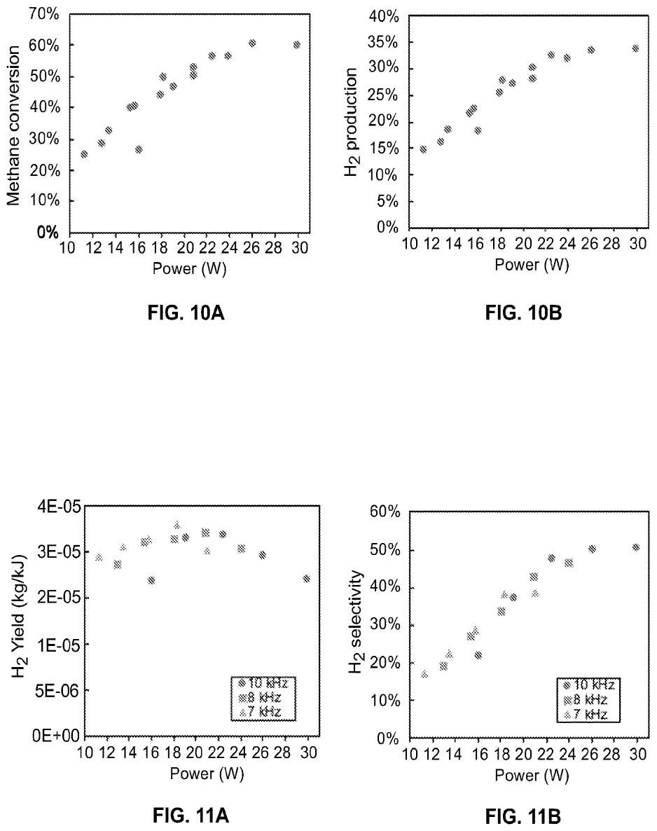

As FIGS. 10A-10B illustrates, at 15.36 W and 15.77 W input powers, on average, the methane conversion is 39.37% and 39.80%, respectively. The difference is due to the input parameters. At the 15.36 W of input power, the pulse frequency repetition is 8 kHz, and the input voltage is 24 kV. At 15.77 W, the PRF is 7 kHz, and the input voltage is 26000 kV. This proves that increasing voltage is more effective in increasing the CH₄ conversion. In addition, increasing the input voltage is more effective than PRF on H₂ production. The H₂ production at a rate of 1.1 L/min and mentioned input voltages and PRF at (a) an input power of 15.77 W is 22.28%, and (b) 15.36 W input power is 21.24%. In other words, higher H₂ production was gained at a higher input power up.

Although increasing power increases the CH₄ conversion and H₂ production, H₂ yields (kg/kJ) were achieved to find the maximum H₂ mass production per unit of input energy. As FIGS. 11A-11B shows, the maximum H₂ yield is at the input power of 18.29 W, input voltage of 28 kV, and PRF of 7 kHz. Also, H₂ selectivity increases by increasing power. The maximum H₂ selectivity is 50.19% at 30 W, 30 KV, and 10 KHz. The H₂ selectivity at maximized H₂ yield is 38.12%.

### Effect of Gas Flow Rate Through the Plasma-Based Reformer

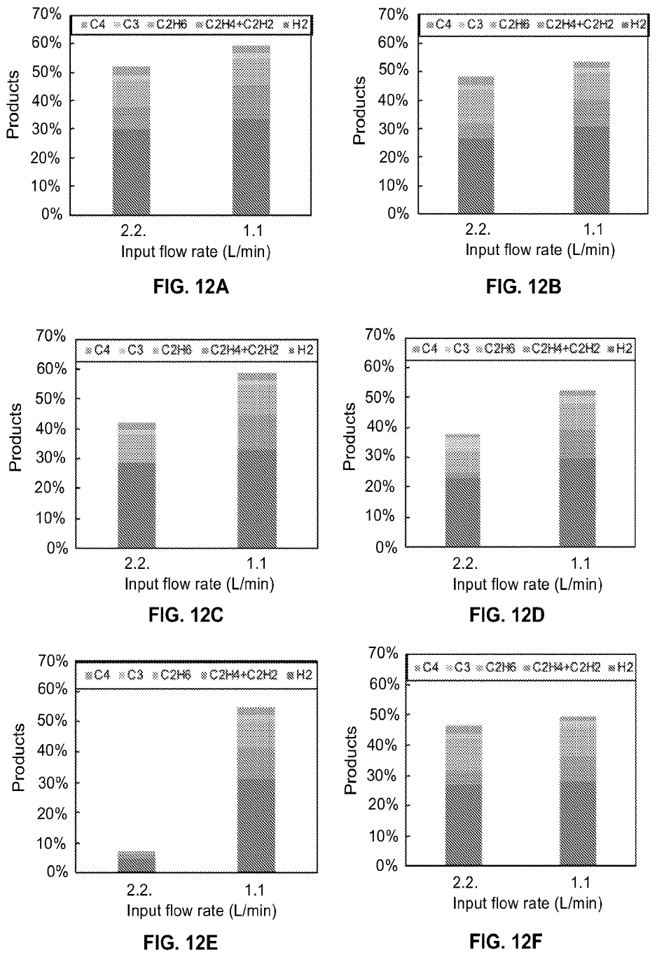

FIGS. 12A-12F show the effect of the gas flow rate on the methane conversion and production of H₂ and co-products. The repetition frequency of 6, 7, 8, and 10 KHz at 26, 28, and 30 kV input voltage are applied. Comparing products versus input flow rate between 1.1 and 2.2 L/min at a fixed voltage and pulse repetition frequency shows that more resistance time results in more H₂ production and CH₄ conversion. It is also understood that at a 2.2 L/min flow rate, increasing the number of pulses per second will increase H₂ production. When the PRF increased by 2 kHz, H₂ production increased by 5.39% and CH₄ conversion by 7.12%. In fact, more pulses in a second are needed at a higher flow rate, which is not in favor of the system's power input. The same results were achieved at lower voltage and frequencies. Therefore, a higher gas flow rate reduces the residence time of reactants in the discharge, which reduces the possibility of collision between electrons and CH₄ molecules and their intermediates subsequently.

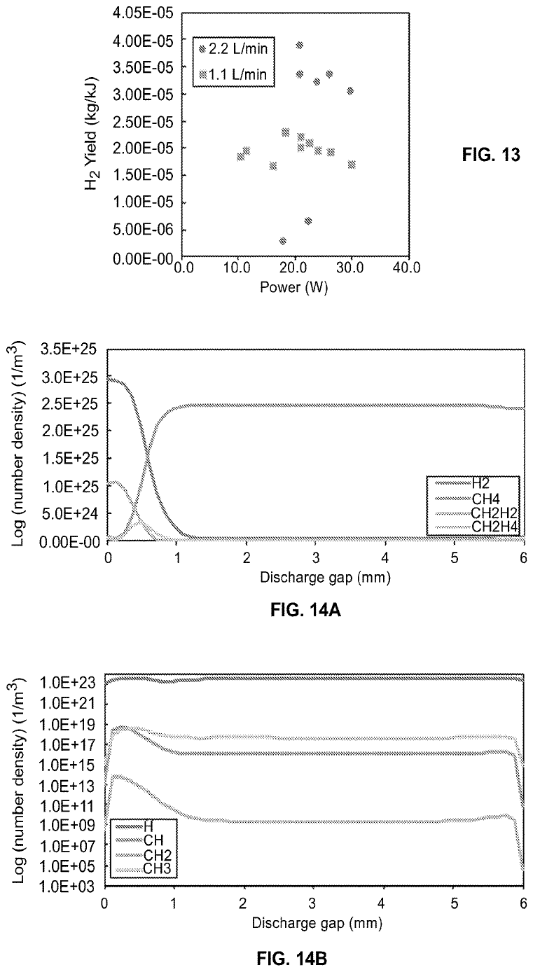

Calculating the H₂ yield (kg/kJ) shows that although the conversion (%) and H₂ production (KJ/kg) will reduce at a higher flow rate, the yield of production in comparison to the input unit of energy will be higher. FIG. 13 shows that when above 20 W and a 28 kV of input voltage is applied, the H₂ yield at 2.2 L/min is higher than 1.1 L/min. Moreover, the analysis shows that with this reformer, when the utilized voltage is less than 20.9 W, the H₂ yield is higher at a lower flow rate (=1.1 L/min). Interestingly, the achieved H₂ yield at around the 22 W power input, as shown in FIG. 13, is varied depending on the input voltage, PRF, and flow-in rates. With this reformer at a flow rate of 2.2 L/min, a minimum of PRF (=7 kHz) and input voltage (=26 kV) are needed to produce hydrogen (~4%). This confirms that at a 2.2 L/min flow, more pulses are needed to reach the maximum H₂ yield with this current setup.

FIG. 13 also shows the maximum H₂ yield achieved at 7 kHz at both flow rates. The maximum H₂ yield is achieved when 30 kV voltage and 7 kHz PRF are applied at a higher rate (2.2 L/min). At a 1.1 L/min flow rate, the maximum H₂ yield was achieved when 28 kV voltage and 7 kHz PRF were applied. In fact, to reach the maximum H₂ yield, higher energy input is required to overcome the reformer's reaction resistance time and molecular velocity.

### Computational Results

A detailed mathematical model is presented in Appendix A to explain the simulation. FIGS. 14A-14B show the logarithm number of densities for primary products and primary radical species at the 30 kV of input voltage and 10 KHz of PRF using COMSOL Multiphysics. The gas velocity through the system was calculated to find the resistance time for reactions. The average velocity of flow-in gas through the system is 1.754 m/s. Hence at a 2.2 L/min flow rate, the resistance time would be 0.00342 s. In the 10 KHz of PRF scenario, thirty-four pulses will be generated from when a molecular leaves anode to reach the cathode. A hybrid model was used to solve the first four nanoseconds of each pulse kinetically for electron impact reactions, and from 4 ns to 0.0001 s, a fluid model was used to solve heavy species reactions. As FIGS. 14A-14B show, the logarithm number of densities of species increased by the positive electrode. Similar to the experimental results, H₂ and C₂H₂ are the dominant components. Among species, H and CH₃ are the primary radical species, and since they are unstable products, they react to produce products including H₂ and C₂H₆ consequently.

What is claimed is:

1. A system for producing hydrogen, comprising:
a gas reformer component;
a membrane-based gas separation component in fluid connection with the gas reformer component; and
means for recirculating non-hydrogen gases in fluid connection with the gas reformer and the membrane-based gas separation component.

2. The system of claim 1, further comprising a storage component in fluid connection with the membrane-based gas separation component.

3. The system of claim 1, wherein the gas reformer is a plasma-based reformer configured to discharge of a plurality of nanosecond pulses of electricity to create a non-thermal plasma.

4. The system of claim 3, wherein each nanosecond pulse of electricity has a pulse width of about 3 ns with an energy input of 3 mJ or less.

5. The system of claim 1, wherein the membrane-based gas separation component comprises a plurality of nanotubes therein configured to flow gases therethrough.

6. The system of claim 1, wherein the means for recirculating non-hydrogen gases is at least one compressor.

7. A method for producing hydrogen, comprising:
a) feeding methane into the plasma-based reformer component of claim 1 to produce a mixture of hydrogen gas and hydrocarbon gases; and
b) passing the mixture through the membrane-based gas separation component to separate the hydrogen gas from the hydrocarbon gases.

8. The method of claim 7, wherein after step b) the method further comprising:
c) flowing the hydrogen gas into a storage container.

9. The method of claim 8, further comprising:
d) flowing the hydrocarbon gases to the plasma-based reformer component; and
e) repeating steps a) to d) at least once.

10. The method of claim 7, wherein the hydrocarbon gases comprise C₂H₂ hydrocarbons, C₂H₆ hydrocarbons or C₃ hydrocarbons or a combination thereof.

11. A modular system for producing hydrogen energy from natural gas, comprising:
in a continuous fluid loop:
a plasma-based reformer operable to reform natural gas into a mixture of hydrogen and low molecular weight hydrocarbon products,
a membrane and separation component operable to separate the mixture into a permeate consisting of the hydrogen and a retentate comprising the low molecular weight hydrocarbons; and
at least one compressor operable to recirculate the retentate to the plasma-based reformer.

12. The modular system of claim 11, further comprising a storage system in fluid connection therewith, said storage system operable to store the hydrogen as an energy source.

13. The modular system of claim 11, wherein the plasma-based reformer comprises a plasma reactor in operable communication with an electric power supply.

14. The modular system of claim 11, wherein the membrane and separation component comprises a plurality of nanotubes operable to effect separation of the mixture.

15. The modular system of claim 11, wherein the retentate comprises C₂H₂ hydrocarbons, C₂H₆ hydrocarbons or C₃ hydrocarbons or a combination thereof.

16. A continuous flow process for producing hydrogen energy, comprising:
a) flowing natural gas into a plasma-based reformer;
b) generating a plasma within the plasma-based reformer to produce hydrogen gas and low molecular weight hydrocarbons from the natural gas;
c) flowing the hydrogen gas and low molecular weight hydrocarbons into a separation component to separate the hydrogen gas from the low molecular weight hydrocarbons, said hydrogen gas flowing through a membrane disposed therein;
d) returning the low molecular weight hydrocarbons retained in the separation component to the plasma-based reformer; and
e) repeating steps a) to d).

17. The continuous flow process of claim 16, further comprising storing the hydrogen gas as an energy source.

18. The continuous flow process of claim 16, wherein the plasma is a non-thermal plasma generated via nanosecond pulses of electricity.

19. The continuous flow process of claim 18, wherein each of the plurality of nanosecond pulses of electricity has a pulse width of about 3 ns and an energy input of 3 mJ or less.

20. The continuous flow process of claim 16, wherein the hydrocarbon gases comprise C₂H₂ hydrocarbons, C₂H₆ hydrocarbons or C₃ hydrocarbons or a combination thereof.
